(19) United States
(12) Patent Application Publication
(10) Pub. No.: US 2023/0011205 A1
(43) Pub. Date: Jan. 12, 2023

(54) ELECTROSURGICAL HANDHELD DEVICE, AND CONTACT BODY FOR AN ELECTROSURGICAL HANDHELD DEVICE

(71) Applicant: OLYMPUS WINTER & IBE GMBH, Hamburg (DE)

(72) Inventors: Martin HORN, Hamburg (DE); Christian BROCKMANN, Hollenstedt (DE)

(73) Assignee: OLYMPUS WINTER & IBE GMBH, Hamburg (DE)

(21) Appl. No.: 17/828,659

(22) Filed: May 31, 2022

Related U.S. Application Data
(60) Provisional application No. 63/208,059, filed on Jun. 8, 2021.

(30) Foreign Application Priority Data
Jun. 9, 2021 (DE) 102021114778.6

(51) Int. Cl. A61B 18/14

(52) U.S. Cl. CPC A61B 18/149; A61B 2018/00982

(57) ABSTRACT

An electrosurgical handheld device and also a contact body for an electrosurgical handheld device, with which both the required cleanliness and the necessary safety can be obtained. This is achieved by the fact that a contact body for an electrosurgical handheld device for receiving an optical guide and for coupling at least one electrical contact of an electrode instrument of the handheld device has an RF cable, which is fixedly connected to the contact body. By way of this RF cable, an RF voltage can be applied to the electrode instrument via the at least one electrical contact. The other end of the RF cable can be connectable to an RF generator.

The invention relates to a contact body for electrosurgical handheld devices according to the preamble of claim 1. The invention also relates to an electrosurgical handheld device according to claim 12.

Electrosurgical handheld devices, for example resectoscopes, are used mainly for endoscopic applications in urology and gynecology, there preferably for treatment in the region of the bladder, the uterus or the prostate. However, the field of use of these instruments is not limited to these regions of the human body and instead also includes the treatment of further organs in the lower part of the human abdomen.

The instruments of the kind described here, for example resectoscopes, have a working element as standard. To treat the diseased tissue, the resectoscope is inserted with an elongate shaft through an opening into the body of the patient. Various medical instruments for treating and/or examining the patient can be arranged in this shaft tube. For example, an electrode to which radiofrequency alternating current can be applied, and which is positioned at a distal end of an electrode carrier, can be inserted into the shaft tube for radiofrequency surgery. For treatments that are to be performed on the patient, for example the cutting of diseased tissue, the electrode carrier with the electrode is arranged on the resectoscope in such way as to be movable relative to the shaft tube and along a shaft axis. The electrode or the tool is assigned to the distal end of the shaft tube.

The electrode carrier is furthermore coupled with its proximal end to the working element, by which it can be moved along the shaft axis. The cutting movement of the electrode is permitted in this way. The working element is usually coupled releasably to the shaft tube. It has a movably mounted contact body, which is also designated as a slide. On this contact body, the electrode carrier can be mechanically and releasably coupled to at least one electrical contact.

The working element is actuated or longitudinally displaced by an operator. For this purpose, the working element is assigned a grip unit with a first grip element and a second grip element. To actuate the working element, the operator grasps the first grip element, and also a second grip element that can have a finger unit or a thumb ring. The first grip element can be fastened to a stationary main body of the working element. The second grip element can be fastened to the contact body.

The movement of the working element takes place counter to a spring tension of a spring, which in the working element of the type in question is designed usually as a leaf spring or as a leg spring. One end of this spring is fastened to the contact body or the slide, and the other end is fastened to an end body or an optical guide plate of a strengthening tube. The nature of the springs or the type of actuation of this spring mechanism depends on whether the working element is an active or passive working element. Whereas the spring in the case of an active working element is designed as a compression spring, in the case of a passive working element it is designed as a tension spring.

The cutting by the electrode is usually effected by a pulling-back movement of the working element. In the case of the active working element, the electrode is for this purpose pulled back (in the proximal direction) against the spring force of the spring. By contrast, in the case of the passive working element, the electrode is first of all pushed forward (in the distal direction) counter to the spring force, in order then to cut through the tissue during the return movement (in the proximal direction) caused by the relaxation of the spring.

An optical unit can also be guided through the shaft of the instruments described here. The rod-like or shaft-like optical unit is guided from the proximal end through a strengthening tube, also called optical guide tube, of the working element into the shaft. Embodiments are known in which the optical unit is guided as a rod lens system or as an optical fiber through the shaft from a proximal end to the distal end. The distal end of the optical unit is directed directly to the surgical region or the site of action of the electrode.

At the proximal end of the optical unit, the operator can observe the treatment through an eyepiece or a camera.

The electrode carrier or the electrode system is connected to the at least one electrical contact of the contact body, and both are then pushed together into or through the shaft. By actuation of the second grip element relative to the first grip element, the contact body together with the electrode carrier can thus be moved to and fro along the shaft axis via the strengthening tube.

In known systems, the strengthening tube is connected fixedly to the working element or the main body at the time of manufacture. At the proximal end of the strengthening tube, the latter is welded to the optical guide plate. Before the strengthening tube is connected to the main body, the contact body has to be pushed over the strengthening tube. For this purpose, the contact body has a corresponding bore parallel to the shaft axis. This bore is dimensioned such that the contact body or the slide can be pushed easily over the strengthening tube.

It is not only the complicated manufacture of the working element that has proven to be particularly disadvantageous, but also the increased effort involved in the repair and replacement of components. Particularly when the contact body is to be replaced for maintenance reasons or because of defects, the strengthening tube has to be removed with difficulty from the main body in order to be able to withdraw the contact body from same.

By way of the mechanical connection between the contact body and the at least one electrical contact of the electrode carrier, the electrode carrier or the electrode can be supplied with electrical energy. For this purpose, the contact body has at least one opening into which at least one electrical contact of the electrode carrier can be guided for the releasable connection. This opening or this blind hole or this bore is designed in such a way that the RF voltage can be applied via an adjoining plug socket in the case of known contact bodies. For this purpose, a plug is usually fitted into the socket and is in turn connectable to an RF generator via a line or a cable. In the case of the known contact bodies or slides, a particular disadvantage has been that the openings or holes and the lumen are difficult to clean. In particular, openings or blind holes for receiving an electrical contact can be cleaned only with considerable effort or not with the required degree of thoroughness. A further problem is that the contact faces between the plug sockets and the cables become worn after repeated use, and the functionality of the electrical connections is thus adversely affected. In individual cases, a poor electrical connection between the electrode instrument and the contact body can lead to electrical flashover or erosion at the contact points. Besides the fact that a defective function of the device means that the treatment cannot be performed, it also poses a safety risk to the persons involved.

Proceeding from this, the object of the invention is to make available an electrosurgical handheld device and a contact body for an electrosurgical handheld device, with which the required cleanliness and also the necessary safety can be obtained.

A contact body for achieving this object has the features of claim 1. Accordingly, provision is made that a contact body for an electrosurgical handheld device for receiving an optical guide and for coupling at least one electrical contact of an electrode instrument of the handheld device has an RF cable, which is fixedly connected to the contact body. By way of this RF cable, an RF voltage can be applied to the electrode instrument via the at least one electrical contact. The other end of the RE cable can be connectable to an RF generator, for example. On account of this fixed connection of the RF cable to the contact body, a corresponding plug socket into which an RF plug is inserted is superfluous. In this way, the number of openings and the lumens in the contact body is reduced. In the present invention, provision can be made that the contact body is designed as a disposable contact body. That is to say, the contact body, exactly like the electrode instrument, is used just for one operation and is then discarded. It is conceivable that the disposable contact body is packaged together with a disposable electrode instrument. With this design of the contact body according to the invention, it is possible to satisfy the requirements of cleanliness and also those of safety.

According to the present invention, provision can also be made that there is a releasable coupling between an element of a grip unit and the contact body. This releasable coupling can be formed on the contact body as at least one coupling means. The at least one coupling means can be releasably coupled, for example, to a first grip element and/or a second grip element of the grip unit. By virtue of this releasable coupling between the contact body and the grip unit or the handheld device, the contact body together with the integral RF cable can be easily coupled to and released from the handheld device. Thus, after a treatment has been completed, the contact body can be released or uncoupled from the handheld device without any great effort, and a new contact body for a further treatment can be connected to the handheld device. This rapid and straightforward exchange does away with the need for complicated cleaning of the contact body.

Moreover, by using a new contact body, it is possible to ensure sufficient safety of the persons involved. For the handling of the contact body, it is additionally conceivable that the latter has an operating element via which the contact body can be gripped particularly efficiently in order to clip it over the optical guide tube or to remove it. For example, this operating element can be an elastically deformable means which is assigned to the contact body.

Preferably, provision is also made according to the invention that the coupling means is formed in the contact body so as to be able to be coupled releasably to an element, in particular the second grip element, of the grip unit. The coupling means is therefore an integral part of the contact body. For example, this coupling means can be a slit-like receptacle or take the form of one or two or more bores into which the second grip element, with corresponding complementary elements, can be releasably connected or latched. This connection can be, for example, a latching connection, a click-in connection, a plug-in connection, a magnetic connection or the like. The second grip element can engage with a latching action in the slit-like receptacle of the contact body. At the end of the slit-like receptacle, which preferably extends over the whole width of the contact body, a widening or bore can be provided in which the second grip element can engage for the releasable connection. It is alternatively conceivable that spring-pretensioned pins of the second grip element engage in receptacles formed as bores in the contact body. Wien the pin tension is relaxed, the second grip element is able to separate from the contact body.

If the electrosurgical handheld device is a resectoscope with a passive working element, this releasable connection between the second grip element and the contact body is already sufficient to allow the handheld device to be used in the intended manner. The releasable connection between said components is designed such that it has the necessary strength to ensure that it is not accidentally released during the treatment. The connection is equally configured in such a way that it can be manually released by the operator without any great force and without the use of a further tool.

It is further conceivable that the slit-like receptacle in the contact body for the connection to the second grip element is oriented perpendicular to a longitudinal axis of the contact body. By virtue of this orientation of the receptacle, the invention described here fits in with the mode of operation of existing grip units which have a spring action parallel to the longitudinal axis of the contact body or of the handheld device.

In an alternative illustrative embodiment of the invention, provision can be made that the coupling means is designed as a latching means, a bayonet catch, a click-in connection, a plug-in connection or another kind of mechanical connection, and the coupling means, which is preferably arranged on a distal end of the contact body, is able to produce a releasable connection to the first grip element of the handheld device. In this illustrative embodiment of the contact body, provision is made that the contact body is connected directly to the first grip element, which is the case for example for an active working element of a resectoscope. By virtue of the above-described mechanical or magnetic coupling, the contact body is thus coupled to the movement of the first grip element. This coupling too can be released and secured in the simple manner described above.

Preferably, provision can also be made according to the invention that the integral RF cable is connected to at least one plug socket, preferably two plug sockets for receiving a respective contact of the electrode instrument, and this at least one plug socket is integrated in the contact body. If the surgical handheld device has an electrode instrument with just one electrical contact, the contact body likewise has only one corresponding receptacle. However, if the electrode instrument has two contacts, for example an active contact and a return contact, the contact body can accordingly have two receptacles for the electrical contacting. These receptacles are likewise arranged parallel to the bore in the contact body and can be designed in the manner of blind holes or can extend through the entire body. The receptacle is connected in the contact body to the integral RF cable. In this way, voltage is supplied directly to the electrode by the RF generator.

Preferably, the invention further provides that the contact body has a slit parallel to a continuous bore. The bore is provided for receiving an optical guide. This optical guide can be designed, for example, as a strengthening tube or as a tubular shaft for receiving a rod-like optical unit. The bore extends from an end face of the contact body to an opposite end face, the bore being oriented parallel to a longitudinal axis of the electrosurgical handheld device. According to the invention, the slit is designed in such a way that the contact body can be plugged over the tubular optical guide, and the optical guide is moved through the slit into the bore. It is equally conceivable that the tubular optical guide can be pressed through the slit into the bore. By virtue of the slit, the contact body can thus also be fastened to a handheld device, or a working element of a handheld device, that has been at least almost finally assembled. Equally, after completion of the treatment, the contact body can be released from the optical guide, specifically without the handheld device needing to be disassembled to any great extent.

In particular, the invention can further provide that the slit extends through the contact body from an outer wall of the contact body as far as the bore, wherein the tubular optical guide can be guided through the slit into the bore. The slit and the bore thus together form a recess in the contact body. Ultimately, the slit constitutes an extension of the interior of the bore. Through this extension, the optical guide can be easily inserted in the bore and also removed again. Provided that the slit-like design of the extension is not too wide, nothing changes as regards the sliding connection between the optical guide and an inner wall of the bore, Rather, the handling of the handheld device remains unaffected by this slit.

In a further advantageous illustrative embodiment of the invention, provision can be made that a plane extending parallel and centrally between the side walls intersects a central axis of the bore. By means of this relative orientation of the side walls and of the bore, the optical guide can be guided in a particularly simple way into the bore. It is additionally conceivable that the aforementioned plane is slightly offset from the central axis. This can facilitate the insertion and removal of the optical guide into and from the bore.

In a particularly advantageous illustrative embodiment of the invention, provision is made that a width of the slit, i.e. a distance between the side walls thereof, is less than the diameter of the bore. The bore can preferably have a diameter of 3 mm to 6 mm, preferably 4 mm to 5 mm, in particular 4.6 mm, and the slit can have a width of 2 mm to 5 mm, preferably 3 mm to 4 mm, in particular 3.5 mm. The diameter of the bore is always slightly greater than the diameter of the tubular optical guide.

A ratio between a width of the slit, in particular a minimum spacing of the side walls of the slit, and a diameter of the bore of 0.6 mm to 0.9 mm, preferably 0.7 mm to 0.8 mm, in particular 0.76 mm, has proven particularly preferable. This ratio of the width and of the diameter is particularly preferable for easy insertion of the optical guide and also for a sufficient sliding resistance of the contact body on the optical guide. If the ratio is too small, there is in particular the risk of the structural parts plastically deforming. If the ratio is too great, the guiding function is no longer ensured. The described values apply for PTFE and may differ for other materials.

The contact body of the surgical handheld device described here can also be designed as a slide of an active or passive resectoscope. It has been found that plastic, in particular PTFE, is particularly advantageous for the contact body on account of the material properties, for example low sliding resistance, high electrical resistance, smooth surface and good processability. However, it is equally conceivable too that the contact body is made of another fluoropolymer, PFA for example. PEEK has also proven advantageous. Another possible illustrative embodiment is one in which the contact body is produced from a reversibly deformable material, which makes the pushing over the optical guide easier. In this case, when installing the contact body, the slit is slightly and flexibly widened. When the contact body is uncoupled, the slit widens again so as to make removal easier.

An electrosurgical handheld device for achieving the object mentioned at the outset has the features of claim 12. Accordingly, provision is made that the handheld device, which can preferably be a resectoscope with an active or passive working element, has an electrode instrument which at a distal end has an electrode and at a proximal end has at least one electrical contact. Moreover, the handheld device has a grip unit with a first grip element and a second grip element. In addition, the instrument has a tubular shaft which is coupled with a proximal end to the first grip element, and an optical guide for receiving an optical unit. This optical guide can be guided through a contact body, wherein the second grip element and a spring are also fastened to this contact body, and has at least one receptacle for an electrical contact of the electrode instrument. According to the invention, this contact body is designed according to at least one of the preceding claims.

A preferred illustrative embodiment of the invention is explained in more detail below with reference to the drawing, in which:

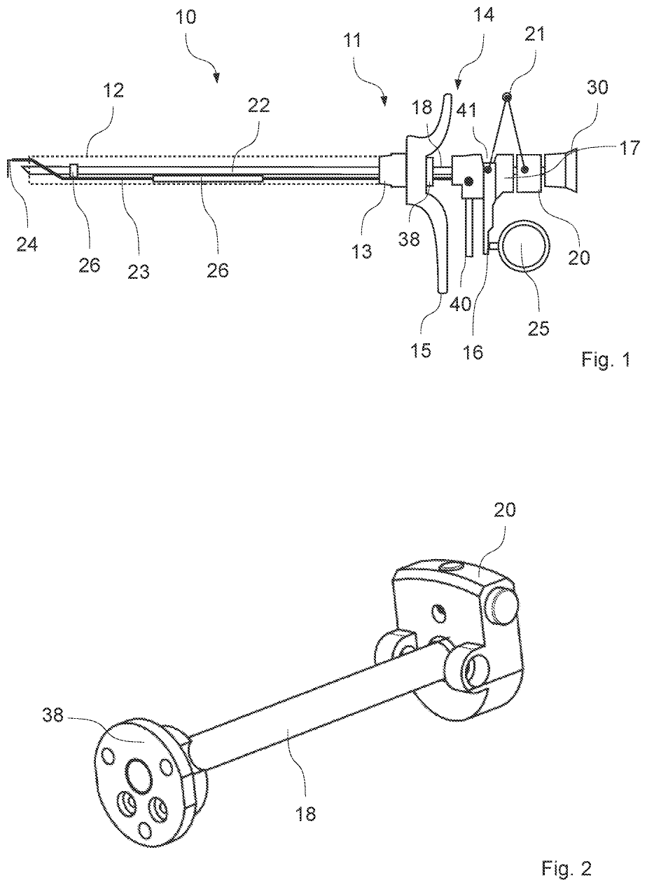

FIG. 1 shows a schematic view of a resectoscope,

FIG. 2 shows a perspective view of an optical guide,

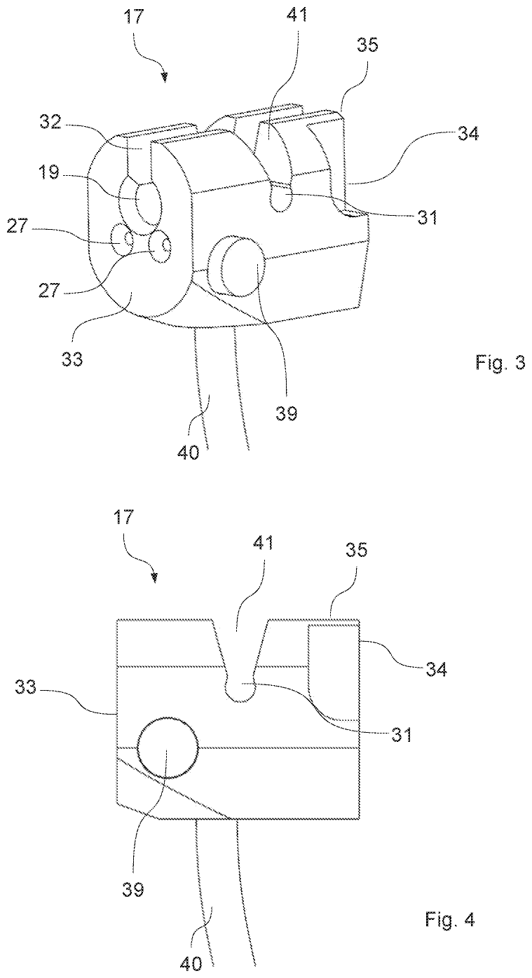

FIG. 3 shows a perspective view of a contact body, and

FIG. 4 shows a side view of the contact body according to FIG. 3.

A possible illustrative embodiment of an electrosurgical handheld device, namely a resectoscope 10, is depicted highly schematically in FIG. 1. The resectoscope 10 has a working element 11 on which an elongate, tubular shaft 12 can be fastened. This shaft 12 is indicated by hatching in FIG. 1 and is fastened with a proximal end to a main body 13 of the working element 11.

The working element 11 has, in addition to the main body 13, a grip unit 14. This grip unit 14 has a first grip element 15 and a second grip element 16. While the first grip element 15 is arranged fixedly on the main body 13, the second grip element 16 is assigned to a contact body 17 in the illustrative embodiment of the working element 11 shown here.

The contact body 17 is guided slidingly on a tubular optical guide 18 or an optical guide tube. For this purpose, the contact body 17 has a bore 19 whose diameter is slightly greater than a diameter of the optical guide 18. Since the contact body 17 can move to and fro on the optical guide 18 along a longitudinal direction of the resectoscope 10 or a longitudinal axis of the shaft 12, the contact body 17 is also designated as a slide.

While the optical guide 18 is connected with a distal end to the main body 13 or an inner tube 22 via an adapter 38 (FIG. 2), an optical guide plate 20 is fastened at a proximal end of the optical guide 18. The tubular optical guide 18 extends through the optical guide plate 20, such that the optical guide 18 is accessible from the proximal direction.

The second grip element 16 and the contact body 17 are connected to the optical guide plate 20 via a spring element 21. This spring element 21 can be a tension spring.

Starting from the main body 13, an inner tube 22 extends in the distal direction. This inner tube 22 can also extend in the proximal direction through the main body 13 and be connected to the optical guide 18. It is equally conceivable that the inner tube 22 and the optical guide 18 are formed in one piece, or that the optical guide 18 extends distally through the main body 13.

An electrode instrument 23 extends parallel to the inner tube 22. This electrode instrument 23 is guided through the main body 13 and with at least one proximal contact is mechanically and releasably coupled in a receptacle 27 to the contact body 17. In the contact body 17, a latching mechanism can be provided which can be released and fixed via a button 39 (FIGS. 3 and 4). The latching mechanism locks the at least one proximal end or the contact of the electrode instrument 23 in the contact body 17. The button 39 or the latching mechanism can be spring-preloaded and can be easily actuated with one finger.

At a distal end, the electrode instrument 23 has an electrode 24. An electrical RF voltage can be applied to this electrode 24. The diseased tissue can be manipulated or cut by means of a plasma that forms at the electrode 24. For this purpose, the operator moves the second gripping means 16, having a thumb ring 25, relative to the first gripping means 15. For stabilizing the electrode instrument 23, the latter can be guided on the inner tube 22 by guides 26.

For applying the RF voltage to the electrode 24, the receptacle 27 of the proximal contact of the electrode instrument 23 can be electrically contacted. For this purpose, the contact body 17 has at least one plug socket (not visible in the figures). This plug socket is in electrical contact with at least one part of an inner wall of the receptacle 27. The contact body 17 or the plug socket is connected in the interior of the contact body 17 to the RF cable 40. The RF cable 40 is integrally connected to the contact body 17. At another end (not shown), the RF cable 40 has a plug with which it is attachable to an RF generator. The contact body 17, together with the RF cable 40, is thus made available or supplied as one unit.

According to the invention, this unit consisting of contact body 17 and RF cable 40 is designed as a disposable contact body 17. In other words, after this operation, this unit is removed from the resectoscope 10 and discarded. For a subsequent operation, a new unit can then be connected to the resectoscope 10.

For performing the intervention, a rod-like optical unit 29 is guided through the inner tube 22 or optical guide 18. A distal end (not visible here) of this optical unit 29 is directed in the direction of the electrode instrument 23, so that the operator has a view of the manipulation of the tissue. This optical unit 29 can be a rod lens system or an optical fiber. As is shown in FIG. 1, an eyepiece 30 or a camera is located at the proximal end of the optical unit 29.

In the manufacture of the working element 11, the assembly of the contact body 17 proves particularly awkward. It was hitherto the case that the optical guide plate 20 was first welded to the optical guide 18, then the contact body 17 was plugged onto the optical guide 18, and then the optical guide 18 was firmly connected by the distal end to the main body 13 or the inner tube 22. For replacement or for maintenance of the contact body 17, these steps had to be repeated in reverse order.

The contact body 17 shown here has a slit 32 (FIG. 3). This slit 32 extends parallel to the bore 19 from an end face 33 to the opposite end face 34 of the contact body 17. The slit 32 is designed in such a way that it extends from an outer wall 35 as far as the bore 19 (FIG. 3). The interior of the bore 19 is thus extended.

In the illustrative embodiment of the slit 32 shown in FIG. 3, it has two parallel side walls 36, 37. The distance between these two side walls 36, 37, i.e. the width of the slit 32, is less than the diameter of the bore 19. Provision is made in particular that a ratio between the width of the slit 32 and the diameter of the bore 19 is 0.6 mm to 0.9 mm, preferably 0.7 mm to 0.8 mm, in particular 0.76 mm.

By virtue of the extension of the bore 19 through the slit 32, it is possible to clip the contact body 17 onto the optical guide 18. For this purpose, the tubular optical guide 18 is guided through the slit 32 into the bore 19. It is conceivable here that an external diameter of the optical guide 18 or the distance between the two side walls 36, 37 deforms for a short time and in a reversible manner. After the assembly of the contact body 17, the grip element 16, the spring element 21 and the electrode instrument 23 can then be connected to the contact body 17.

FIG. 4 shows a further essential feature of the invention. The receptacle 41, designed as a slit or wedge, serves to couple the second grip element 16 to the contact body 17 in a simple and reliable manner. For this purpose, corresponding bearing pins of the grip element 16 are pressed into the receptacle 41 from above. This coupling can be released again by the action of a slight force. Alternatively, it is likewise conceivable that the contact body 17 has only two bores 31 into which two spring-pretensioned bearing pins of the second grip element 16 can be inserted with a latching action. In a further illustrative embodiment (not shown), provision can be made that the second grip element 16 is coupled magnetically to the contact body 17. By virtue of this releasable coupling between the grip element 16 and the contact body 17, a quick and simple connection can be effected between said components.

### LIST OF REFERENCE SIGNS

10 resectoscope
11 working element
12 shaft
13 main body
14 grip unit
15 first grip element
16 second grip element
17 contact body
18 optical guide
19 bore
20 optical guide plate
21 spring element
22 inner tube
23 electrode instrument
24 electrode
25 thumb ring
26 guide
27 receptacle
29 optical unit
30 eyepiece
31 bore
32 slit
33 end face
34 end face
35 outer wall
36 side wall
37 side wall
38 adapter
39 button
40 RF cable
41 receptacle

1. A contact body for an electrosurgical handheld for receiving an optical guide and for coupling at least one electrical contact of an electrode instrument of the handheld device, wherein an RF cable which is fixedly connected to the contact body and by which an RF voltage can be applied to the electrode instrument via the at least one electrical contact.

2. The contact body for an electrosurgical handheld device as claimed in claim 1, wherein a releasable coupling to at least one element of a grip unit of the handheld device, the contact body having at least one coupling means for the releasable coupling.

3. The contact body for an electrosurgical handheld device as claimed in claim 2, wherein the coupling means is designed as a receptacle in the contact body, to which receptacle an element of the grip unit is able to be releasably coupled.

4. The contact body for an electrosurgical handheld device as claimed in claim 3, wherein the receptacle is designed like a slit or as a bore, and the second grip element is connectable to the receptacle with a latching action, or in that the receptacle is magnetic, for the purpose of magnetic coupling to the second grip element.

5. The contact body for an electrosurgical handheld device as claimed in claim 1, wherein the receptacle is oriented perpendicular to a longitudinal axis of the contact body.

6. The contact body for an electrosurgical handheld device as claimed in claim 2, wherein the coupling means is designed as a latching means, a bayonet catch, a click-in connection or another kind of mechanical connection, and the coupling means, which is arranged on a distal end of the contact body, is able to produce a releasable connection to the first grip element of the handheld device.

7. The contact body for an electrosurgical handheld device as claimed in claim 1, wherein the RF cable is connected to at least one plug socket for receiving a respective contact of the electrode instrument, and this at least one plug socket is integrated in the contact body.

8. The contact body for an electrosurgical handheld device as claimed in claim 1, wherein a slit which is oriented parallel to a continuous bore, for receiving the optical guide through the contact body, and parallel to a longitudinal axis of the contact body.

9. The contact body for an electrosurgical handheld device as claimed in claim 8, wherein the slit extends from an outer wall of the contact body as far as the bore, the tubular optical guide being able to be guided through the slit into the bore.

10. The contact body for an electrosurgical handheld device as claimed in claim 1, wherein the contact body is a slide for an active or passive resectoscope.

11. The contact body for an electrosurgical handheld device as claimed in claim 1, wherein the contact body is made of plastic.

12. An electrosurgical handheld device with an electrode instrument which at a distal end has an electrode and at a proximal end has at least one electrical contact, with a grip unit consisting of a first grip element and a second grip element, with a tubular shaft which is coupled with a proximal end to the first grip element, with an optical guide for receiving an optical unit, and a contact body through which the optical guide is able to be guided, the second grip element being able to be fastened, and in which the at least one electrical contact of the electrode instrument can be latched and/or electrically contacted, wherein a contact body as claimed in claim 1.
